(19) United States
(12) Patent Application Publication
(10) Pub. No.: US 2005/0124595 A1
(43) Pub. Date: Jun. 9, 2005

(54) Compositions and methods for treating glaucoma and ocular hypertension

(76) Inventors: Michael A Goetz, Scotch Plains, NJ (US); Gregory J Kaczorowski, Edison, NJ (US)

Correspondence Address: MERCK AND CO., INC, P O BOX 2000, RAHWAY, NJ, 07065-0907, US

(21) Appl. No.: 10/505,580

(22) PCT Filed: Mar. 11, 2003
(86) PCT No.: PCT/US03/07202

Related U.S. Application Data
(60) Provisional application No. 60/364,926, filed on Mar. 15, 2002.

(51) Int. Cl. A61K 31/58

(52) U.S. Cl. 514/176; 514/410

(57) ABSTRACT

This invention relates to the use of potent potassium channel blockers or a formulation thereof in the treatment of glaucoma and other conditions related to elevated intraoccular pressure in the eye of a patient. This invention also relates to the use of such compounds to provide a neuroprotective effect to the eye of a mammalian species, particularly humans.

This application claims the benefit of U.S. provisional application 60/364,926, filed Mar. 15, 2002.

### BACKGROUND OF THE INVENTION

Glaucoma is a degenerative disease of the eye wherein the intraocular pressure is too high to permit normal eye function. Damage eventually occurs to the optic nerve head, resulting in irreversible loss of visual function. If untreated, glaucoma may eventually lead to blindness. Elevated intraocular pressure or ocular hypertension, is now believed by the majority of ophthalmologists to represent the earliest phase in the onset of glaucoma.

Many of the drugs formerly used to treat glaucoma proved unsatisfactory. The early methods of treating glaucoma employed pilocarpine and produced undesirable local effects that made this drug, though valuable, unsatisfactory as a first line drug. More recently, clinicians have noted that many β-adrenergic antagonists are effective in reducing intraocular pressure. While many of these agents are effective for this purpose, there exist some patients with whom this treatment is not effective or not sufficiently effective. Many of these agents also have other characteristics, e.g., membrane stabilizing activity, that become more apparent with increased doses and render them unacceptable for chronic ocular use and can also cause cardiovascular effects.

Although pilocarpine and β-adrenergic antagonists reduce intraocular pressure, none of these drugs manifests its action by inhibiting the enzyme carbonic anhydrase, and thus they do not take advantage of reducing the contribution to aqueous humor formation made by the carbonic anhydrase pathway.

Agents referred to as carbonic anhydrase inhibitors decrease the formation of aqueous humor by inhibiting the enzyme carbonic anhydrase. While such carbonic anhydrase inhibitors are now used to treat intraocular pressure by systemic and topical routes, current therapies using these agents, particularly those using systemic routes are still not without undesirable effects. Because carbonic anhydrase inhibitors have a profound effect in altering basic physiological processes, the avoidance of a systemic route of administration serves to diminish, if not entirely eliminate, those side effects caused by inhibition of carbonic anhydrase such as metabolic acidosis, vomiting, numbness, tingling, general malaise and the like. Topically effective carbonic anhydrase inhibitors are disclosed in U.S. Pat. Nos. 4,386,098; 4,416,890; 4,426,388; 4,668,697; 4,863,922; 4,797,413; 5,378,703, 5,240,923 and 5,153,192.

Prostaglandins and prostaglandin derivatives are also known to lower intraocular pressure. U.S. Pat. No. 4,883,819 to Bito describes the use and synthesis of PGAs, PGBs and PGCs in reducing intraocular pressure. U.S. Pat. No. 4,824,857 to Goh et al. describes the use and synthesis of PGD2 and derivatives thereof in lowering intraocular pressure including derivatives wherein C-10 is replaced with nitrogen. U.S. Pat. No. 5,001,153 to Ueno et al. describes the use and synthesis of 13,14-dihydro-15-keto prostaglandins and prostaglandin derivatives to lower intraocular pressure. U.S. Pat. No. 4,599,353 describes the use of eicosanoids and eicosanoid derivatives including prostaglandins and prostaglandin inhibitors in lowering intraocular pressure.

Prostaglandin and prostaglandin derivatives lower intraocular pressure by increasing uveoscleral outflow. This is true for both the F type and A type of Pgs and hence presumably also for the B, C, D, E and J types of prostaglandins and derivatives thereof. A problem with using prostaglandin derivatives to lower intraocular pressure is that these compounds often induce an initial increase in intraocular pressure, can change the color of eye pigmentation and cause proliferation of some tissues surrounding the eye.

As can be seen, there are several current therapies for treating glaucoma and elevated intraocular pressure, but the efficacy and the side effect profiles of these agents are not ideal. Recently potassium channel blockers were found to reduce intraocular pressure in the eye and therefore provide yet one more approach to the treatment of ocular hypertension and the degenerative ocular conditions related thereto. Blockage of potassium channels can diminish fluid secretion, and under some circumstances, increase smooth muscle contraction and would be expected to lower IOP and have neuroprotective effects in the eye. (see U.S. Pat. Nos. 5,573,758 and 5,925,342; Moore, et al., Invest. Ophthalmol. Vis. Sci 38, 1997; WO 89/10757, WO94/28900, and WO 96/33719).

Indole diterpenes are known maxi-K blockers and many are tremorgenic. Indole diterpenes are known to lower intraocular pressure. See U.S. Pat. No. 5,573,758. See also U.S. Ser. No. 09/765,716, filed Jan. 17, 2001, incorporated herein by reference.

### SUMMARY OF THE INVENTION

This invention relates to novel potent potassium channel blockers or a formulation of the maxi-K channel blockers thereof in the treatment of glaucoma and other conditions which are related to elevated intraocular pressure in the eye of a patient. This invention also relates to the use of such compounds to provide a neuroprotective effect to the eye of mammalian species, particularly humans. More particularly this invention relates to the treatment of glaucoma and ocular hypertension (elevated intraocular pressure) using indole diterpene compounds having structural formulas I-III: 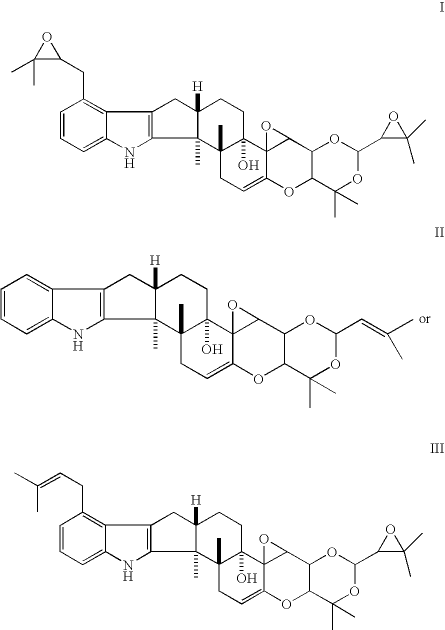
or a pharmaceutically acceptable salt, enantiomer, diastereomer or mixture thereof.

The claimed compounds lack the tremorgenic liability of other indole diterpenes, yet retain excellent potency against the maxi-K channel.

### DETAILED DESCRIPTION OF THE INVENTION

The present invention is directed to novel indole diterpenes of formula I-III described above. The compounds of this invention block the maxi-K channel and do not have a tremogenic effect. This invention is also directed to a method for treating ocular hypertension or glaucoma which comprises administering to a patient in need of such treatment a therapeutically effective amount of a compound having a structural formula: 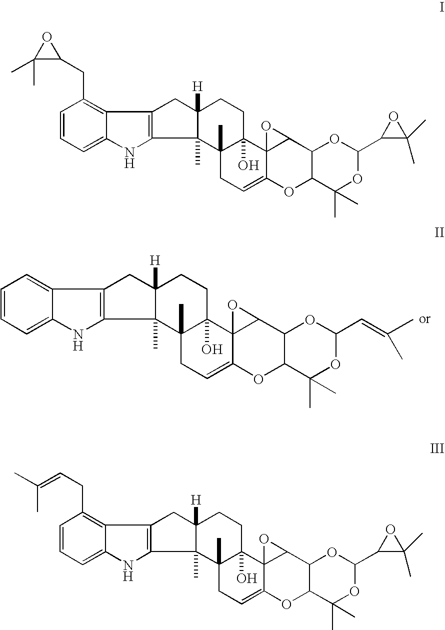
or a pharmaceutically acceptable salt, enantiomer, diastereomer or mixture thereof.

Another embodiment of the invention is the method described above wherein the compound of formula I, II or III is applied as a topical formulation.

Yet another embodiment contemplates the method described above wherein the topical formulation is a solution or suspension.

And yet another embodiment is the method described above, which comprises administering a second active ingredient, concurrently or consecutively, wherein the second active ingredient is selected from a β-adrenergic blocking agent, a parasympathomimetic agent, a carbonic anhydrase inhibitor, and a prostaglandin or a prostaglandin derivative thereof.

Another embodiment is the method described above wherein the β-adrenergic blocking agent is timolol; the parasympathomimetic agent is pilocarpine; the carbonic anhydrase inhibitor is dorzolamide, acetazolaride, metazolamide or brinzolamide; the prostaglandin is latanoprost or rescula, and the prostaglandin derivative is a hypotensive lipid derived from PGF2α prostaglandins.

A further embodiment is a method for treating macular edema or macular degeneration which comprises administering to a patient in need of such treatment a pharmaceutically effective amount of a compound of structural formula I, II or III: 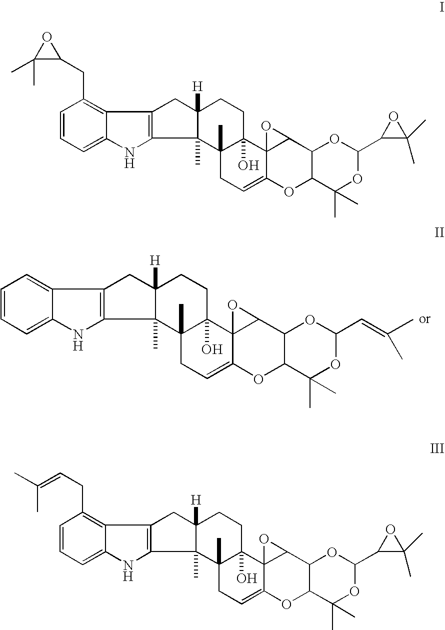
or a pharmaceutically acceptable salt, enantiomer, diastereomer or mixture thereof.

Another embodiment is the method described above wherein the compound of formula I, II or III is applied as a topical formulation.

A further embodiment is illustrated by a method for increasing retinal and optic nerve head blood velocity or increasing retinal and optic nerve oxygen tension which comprises administering to a patient in need of such treatment a therapeutically effective amount of a compound of Formula I, II or III: 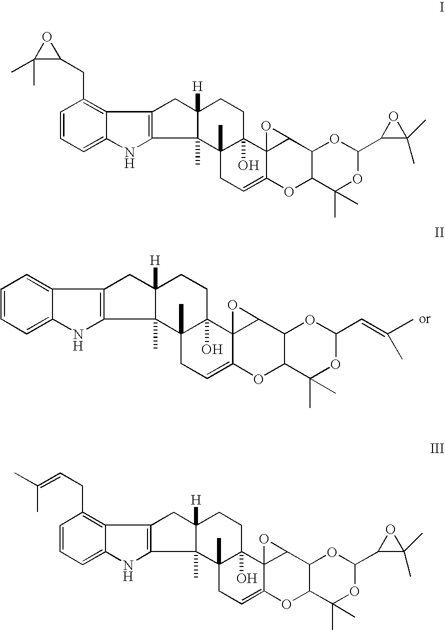
or a pharmaceutically acceptable salt, enantiomer, diastereomer or mixture thereof.

And another embodiment is the method described above wherein the compound of formula I, II or III is applied as a topical formulation.

Another embodiment of the invention is a method for providing a neuroprotective effect to a mammalian eye which comprises administering to a patient in need of such treatment a therapeutically effective amount of a compound of Formula I, II or III: 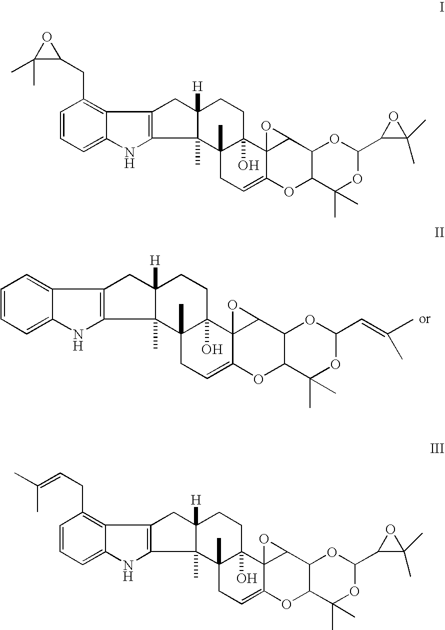
or a pharmaceutically acceptable salt, enantiomer, diastereomer or mixture thereof.

Also within the scope of the invention is the method described above wherein the compound of Formula I, II or III is applied as a topical formulation.

Also contemplated to be within the scope of the present invention is the topical formulation of Compound I, II or III as described above wherein the topical formulation also contains xanthan gum or gellan gum.

The invention is described herein in detail using the terms defined below unless otherwise specified.

This invention is also concerned with a method of treating ocular hypertension or glaucoma by administering to a patient in need thereof one of the compounds of formula I in combination with a β-adrenergic blocking agent such as timolol, a parasympathomimetic agent such as pilocarpine, carbonic anhydrase inhibitor such as dorzolamide, acetazolamide, metazolamide or brinzolamide, a prostaglandin such as latanoprost, rescula, S1033 or a prostaglandin derivative such as a hypotensive lipid derived from PGF2α prostaglandins. An example of a hypotensive lipid (the carboxylic acid group on the α-chain link of the basic prostaglandin structure is replaced with electrochemically neutral substituents) is that in which the carboxylic acid group is replaced with a C₁₋₆ alkoxy group such as OCH₃ (PGF₂ₐ 1-OCH₃), or a hydroxy group (PGF₂ₐ 1-OH).

Preferred potassium channel blockers are calcium activated potassium channel blockers. More preferred potassium channel blockers are high conductance, calcium activated potassium (maxi-K) channel blockers. Maxi-K channels are a family of ion channels that are prevalent in neuronal, smooth muscle and epithelial tissues and which are gated by membrane potential and intracellular Ca²+.

Intraocular pressure (IOP) is controlled by aqueous humor dynamics. Aqueous humor is produced at the level of the non-pigmented ciliary epithelium and is cleared primarily via outflow through the trabecular meshwork. Aqueous humor inflow is controlled by ion transport processes. It is thought that maxi-K channels in non-pigmented ciliary epithelial cells indirectly control chloride secretion by two mechanisms; these channels maintain a hyperpolarized membrane potential (interior negative) which provides a driving force for chloride efflux from the cell, and they also provide a counter ion (K+) for chloride ion movement. Water moves passively with KCl allowing production of aqueous humor. Inhibition of maxi-K channels in this tissue would diminish inflow. Maxi-K channels have also been shown to control the contractility of certain smooth muscle tissues, and, in some cases, channel blockers can contract quiescent muscle, or increase the myogenic activity of spontaneously active tissue. Contraction of ciliary muscle would open the trabecular meshwork and stimulate aqueous humor outflow, as occurs with pilocarpine. Therefore maxi-K channels could profoundly influence aqueous humor dynamics in several ways; blocking this channel would decrease IOP by affecting inflow or outflow processes or by a combination of affecting both inflow/outflow processes.

The present invention is based upon the finding that maxi-K channels, if blocked, inhibit aqueous humor production by inhibiting net solute and H₂O efflux and therefore lower IOP. This finding suggests that maxi-K channel blockers are useful for treating other ophthamological dysfunctions such as macular edema and macular degeneration. It is known that lowering of IOP promotes increased blood flow to the retina and optic nerve. Accordingly, this invention relates to a method for treating macular edema, macular degeneration or a combination thereof.

Additionally, macular edema is swelling within the retina within the critically important central visual zone at the posterior pole of the eye. An accumulation of fluid within the retina tends to detach the neural elements from one another and from their local blood supply, creating a dormancy of visual function in the area.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Ophthalmol. Vol. 112, January 1994, pp. 37-44; Investigative Ophthamol. & Visual Science, 32, 5, April 1991, pp. 1593-99. It is believed that maxi-K channel blockers are also useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, increasing retinal and optic nerve oxygen tension as well as providing a neuroprotective effect or a combination thereof.

The maxi-K channel blockers used are preferably administered in the form of ophthalmic pharmaceutical compositions adapted for topical administration to the eye such as solutions, ointments, creams or as a solid insert. Formulations of this compound may contain from 0.01 to 5% and especially 0.5 to 2% of medicament. Higher dosages as, for example, about 10% or lower dosages can be employed provided the dose is effective in reducing intraocular pressure, treating glaucoma, increasing blood flow velocity or oxygen tension or providing a neuroprotective effect. For a single dose, from between 0.001 to 5.0 mg, preferably 0.005 to 2.0 mg, and especially 0.005 to 1.0 mg of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a micro-particle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mammalian eye will be about once or twice daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The maxi-K channel blockers used in the present invention are made by a microbiological processes employing the strain Chaunopycnis pustulata. (MF 5785), previously described and identified as Nalanthamala sp. in U.S. Pat. No. 5,541,208, herein incorporated by reference. This strain, ATCC 74192, is available from the American Type Culture Collection located at 12301 Parklawn Drive in Rockville, Md.

The maxi-K channel blockers used in the present invention can also made by a microbiological processes employing the strain Chaunopycnis pustulata. (MF6885). This strain will be deposited at the American Type Culture Collection as ATCC PTA-4133, located at 10801 University Blvd., Manassas, Va. 20110-2209. This novel strain and its use to make the maxi-K channel blockers of formula I, II and II is another aspect of this invention.

The compounds used in the present invention can be made by a fermentation process for producing potassium channel antagonists comprising:
(a) inoculating seed medium (Table 1) with mycelia and conidia of Chaunopycnis pustulata MF5785 (ATCC 74192) or MF6885 (ATCC PTA-4133);
(b) incubating the inoculated fungal fermentation at room temperature (20-30° C.) under humid conditions with constant fluorescent light, preferably with shaking, most preferably on a rotary shaker with a 5-cm throw at 220 rpm;
(c) using the culture produced in step (b) to inoculate a liquid production medium and further incubating under the conditions defined in step (b) to produce Compounds I, II, and III.

Maximal accumulation of compounds in the fermentation broth occurs between 7-11 days. The invention further comprises a step (d) in which the compounds produced in the fermentation broth under suitable defined and controlled conditions are purified and isolated from the broth. Suitable isolation procedures include, for example, extraction of the culture medium with an alcoholic or oxygenated solvent, such as an ether or ketone, preferably methylethylketone.

The strains MF5785 and MF6885 have been identified and described in detail as Chaunopycnis pustulata (Bills, G. F., J. D. Polishook, M. A. Goetz, R. F. Sullivan, & J. F. White, Jr. 2002. Chaunopycnis pustulata sp. nov., a new clavicipitalean anamorph producing metabolites that modulate potassium ion channels. Mycological Progress 1:3-15). The strain MF5785 was isolated from unidentified twigs collected in the province of Nuevo Leon, Mexico. The strain MF6885 was isolated from soil of dunes under Pinus pinea, collected in the province of Cádiz, Spain near Conil de la Frontera. These organisms grow well and sporulate abundantly in most mycological media. In agar culture, the strains exhibit the following morphological features:

Colonies on Sabouraud's maltose agar attaining 14-18 mm in diam in 14 d, cottony, dense, sulcate, slightly umbonate, margin even, hyaline at the margin, soon white to pale yellow. Reverse light yellow (Warm Buff, Light Ochraceous-Buff, 4A5, 4B5). No exudates or occasionally exuding a clear sparse liquid, soluble pigments in the agar absent. The colors used to describe colonies etc are standard colors: according to Ridgway, R. (1912). Color Standards and Nomenclature. Washington, D.C., U.S.A., Published by the author.

Colonies on cornmeal agar attaining 21-22 mm in diam in 14 d, appressed, margin even, hyaline, powdery or farinaceous from conidial pustules, conidial pustules light pink (Light Pink, Chatenay Pink, 10A2, 10A3), dull gray after one month. Reverse hyaline. No exudates or soluble pigments.

Colonies on YM agar attaining 15 mm in diam in 14 d, dense, radially sulcate, umbonate, white toward margin, becoming light pink, light grayish pink as conidia mature (Pinkish Vinaceous, Pale Grayish Vinaceous, Pale Vinaceous-Fawn, Pale Brownish Vinaceous, 10B2, 10B3, 1B2, 11B3), in age becoming to grayish vinaceous, pale gray to gray (Light Drab, Pale Mouse Gray, Gull Gray, 6C2, 6C3) as entire colony surface is dominated by mature conidial masses. Reverse translucent to pale yellow. Exudates sparse, clear, no soluble pigments. No growth at 37 C.

Colonies on brain-heart infusion agar attaining 11 mm in diam in 14 d, appressed, with little aerial mycelium, radially rivulose, white, to pale yellow (Naples Yellow, Straw Yellow, 4A4, 4A5), margin even. Reverse pale yellow.

Conidiomata absent, or pustular to sporodochial, forming discrete pustules on weak media, e.g., water agar or cornmeal agar, or as dense, irregular, confluent sporodochia on more nutrient-rich media, e.g., YM agar (YM agar: purchased from Difco and used according to directions. Contains 3 g yeast extract, 3 g malt extract, 5 g peptone, 10 g glucose and 20 g agar per liter of water). Conidiophores micronematous, occasionally semi-micronematous, integrated, up to 30 μm tall, but usually 6-12 μm tall, branched or not, septate or not, often only a simple right-angle branch from main hyphal axis, often aggregated in irregular groups on right-angle branched hyphae, or in short rows, but not in a penicillate arrangement, usually with a single terminal conidiogenous locus, but occasionally conidiogenous loci are lateral or intercalary, in mature colonies aggregated into Trichoderma-like tufts, in old cultures forming irregular, confluent sporodochia. Conidiogenous cells terminal or intercalary, phialidic, cylindrical to lageniform, 4-10 μm long, when mature inflated at the base, 2-4 μm wide. Conidia hyaline, thin-walled, broadly ellipsoidal or obovate, with a slightly flattened base, 2-3(−5)×1.5-2.5 μm, accumulating in dry chains, sometime with faint connectives evident. Hyphae septate, branched, finely incrusted in mature regions of the colonies.

Additional information on strain MF5785, (ATCC 74192) and MF6885, (ATTC PTA-4133) can be found in Bills, G. F., J. D. Polishook, M. A. Goetz, R. F. Sullivan, & J. F. White, Jr. 2002. Chaunopycnis pustulata sp. nov., a new clavicipitalean anamorph producing metabolites that modulate potassium ion channels. Mycological Progress 1:3-15. The sequences of their 28s ribosomal DNA and the intertranscribed spacers of the ribosomal DNA can also be used to characterize these strains and differentiate them from other similar fungi. Those sequences have been deposited in the National Center for Biotechnology Information (GenBank) under accession numbers AF389194 (ribosomal DNA intertranscribed spacer region of MF5785), AF373283 (28S ribosomal DNA of MF5785), AF389189 8 (ribosomal DNA intertranscribed spacer region of MF6885), and AF389190 (28S ribosomal DNA of MF6885).

In general, Compounds I, II, and III can be produced by culturing (fermenting) strain MF5785 (ATCC 74192) or MF6885 (ATCC PTA-4133) in an aqueous nutrient medium containing assimilable carbon and nitrogen sources, preferably under submerged aerobic conditions, and shaking the culture under constant fluorescent light, preferably 450 to 700 nm, until substantial amounts of Compounds I, II, and III is detected in the fermentation broth. The culture is incubated in a aqueous medium at a temperature between 20° C. and 37° C., preferably 25° C. for a period of time necessary to complete the formation of Compounds I, II, and III, usually for a period between 3 to 28 days, preferably between 7 to 11 days, preferably on a shaking means, most preferably on a rotary shaker operating at 220 rpm with a 5 cm throw. The aqueous production medium is maintained at a pH between 5 and 8, preferably about 6.0, at the initiation and termination (harvest) of the fermentation process. The desired pH may be maintained by the use of a buffer such as [2-(N-morpholino)ethanesulfonic acid] monohydrate (MES), 3-(N-morpholino)propanesulfonic acid (MOPS), phosphate buffer or any other buffer effective in pH 5 to 8, or by choice of nutrient materials which inherently possess buffering properties, such as production media described herein below. The active compounds are extracted from the mycelial growth of the culture is with a suitable solvent, such as alcoholic or oxygenated solvent such as an ester or ketone. The preferred solvent for extraction is methylethylketone (MEK). The solution containing the desired compound is concentrated and then subjected to chromatographic separation to isolate compounds I, II and III from the cultivation medium.

The preferred sources of carbon in the nutrient medium include sucrose, glucose, fructose, mannitol, glycerol, xylose, galactose, lactose, sorbitol, starch, dextrin, other sugars and sugar alcohols, starches and other carbohydrates, or carbohydrates derivatives, and the like. Other sources which may be included are maltose, rhamnose, raffinose, arabinose, mannose, salicin, sodium succinate, acetate, and the like as well as complex nutrients such as yellow corn meal, oat flour, millet, rice, cracked corn, and the like. The exact quantity of the carbon source which is utilized in the medium will depend, in part, upon the other ingredients in the medium, but it is usually found that an amount of carbohydrate between 0.5 and 15 percent by weight of the medium is satisfactory. These carbon sources can be used individually or several such carbon sources may be combined in the same medium.

The preferred sources of nitrogen are yeast extract, yellow corn meal, meat extract, peptone, gluten meal, cottonseed meal, soybean meal and other vegetable meals (partially or totally defatted), casein hydrolysates, soybean hydrolysates and yeast hydrolysates, corn steep liquor, dried yeast, wheat germ, feather meal, peanut powder, distiller's solubles, etc., as well as inorganic and organic nitrogen compounds such as ammonium salts (e.g. ammonium nitrate, ammonium sulfate, ammonium phosphate, etc.), urea, amino acids such as methionine, phenylalanine, serine, alanine, proline, glycine, arginine or threonine, and the like. The various sources of nitrogen can be used alone or in combination in amounts ranging from 0.2 to 10 percent by weight of the medium.

The carbon and nitrogen sources, though advantageously employed in combination, need not be used in their pure form because less pure materials which contain traces of growth factors and considerable quantities of mineral nutrients are also suitable for use. When desired, there may be added to the medium inorganic salts, sodium, potassium, magnesium, calcium, phosphate, sulfate, chloride, carbonate, and like ions which can be incorporated in the culture medium as sodium or calcium carbonate, sodium or potassium phosphate, sodium or potassium chloride, sodium or potassium iodide, magnesium salts, copper salts, cobalt salts, and the like. Also included are trace metals such as cobalt, manganese, iron, molybdenum, zinc, cadmium, copper, and the like. The various sources of inorganic salts can be used alone or in combination in amounts ranging from 0.1 to 1.0, and trace elements ranging from 0.001 to 0.1 percent by weight of the medium.

If necessary, especially when the culture medium foams seriously, a defoaming agent, such as polypropylene glycol 2000 (PPG-2000), liquid paraffin, fatty oil, plant oil, mineral oil or silicone may be added.

Submerged aerobic fermentation conditions in fermentors are preferred for the production of Compounds I, II, and III in large amounts. For the production in small amounts, a shaking or surface culture in a flask or bottle is employed. Furthermore, when the growth is carried out in large tanks, it is preferable to use the vegetative form of the organism for inoculation in the production tanks in order to avoid growth lag in the process of production of Compounds I, II, and III.

Accordingly, it is desirable first to produce a vegetative inoculum of the organism by inoculating a relatively small quantity of culture medium with spores or mycelia of the organism produced in a “slant,” or from previously prepared frozen mycelia, and culturing the inoculated medium, also called the “seed medium”, and then aseptically transferring the cultured vegetative inoculum to large tanks. The seed medium, in which the inoculum is produced may be seen in Table 1 and is generally autoclaved to sterilize the medium prior to inoculation. The seed medium is generally adjusted to a pH between 5 and 8, preferably about 6.8, prior to the autoclaving step by suitable addition of an acid or base, preferably in the form of a dilute solution of hydrochloric acid or sodium hydroxide. Growth of the culture in this seed medium is maintained between 26° C. and 37° C., preferably 25° C. Incubation of culture MF5785 (ATCC 74192) in a seed medium, preferably that in Table 1, is usually conducted for a period of about 2 to 6 days, preferably 3 to 4 days, with shaking, preferably on a rotary shaker operating at 220 rpm with a 5 cm throw; the length of incubation time may be varied according to fermentation conditions and scales. If appropriate, a second stage seed fermentation may be carried out in the seed medium (Table 1) for greater production of mycelial mass by inoculating fresh seed medium with a portion of the culture growth and then incubating under similar conditions but for a shortened period. The resulting growth then may be employed to inoculate, a production medium, preferably the Liquid Production Medium (Table 2). The fermentation liquid production medium inoculated with the seed culture growth is incubated for 3 to 28 days, usually 7 to 11 days, with agitation. Agitation and aeration of the culture mixture may be accomplished in a variety of ways. Agitation may be provided by a propeller or similar mechanical agitation equipment, by revolving or shaking the fermentation mixture within the fermentor, by various pumping equipment or by the passage of sterile air through the medium. Aeration may be effected by passing sterile air through the fermentation mixture.

Preferred seed and production media for carrying out the fermentation include the following media:

TABLE 1
Seed Medium

| | | Trace Element Mix | |
|---|---|---|---|
| | per liter | | per liter |
| Corn Steep Liquor | 5 g | FeSO₄.H₂O | 1 g |
| Tomato Paste | 40 g | MnSO₄.4H₂O | 1 g |
| Oat flour | 10 g | CuCl₂.2H₂O | 25 mg |
| Glucose | 10 g | CaCl₂ | 100 mg |
| Trace element mix 10 mL | | H₃BO₃ | 56 mg |
| | | (NH₄)₆Mo₇O₂₄.4H₂O | 19 mg |
| pH = 6.8 | | ZnSO₄.7H₂O | 200 mg |

TABLE 2
Liquid Production Medium

| Component | Per Liter |
|---|---|
| Yellow Cornmeal | 50.0 g |
| Yeast Extract | 1.0 g |
| Sucrose | 80.0 g |
| Distilled Water | 1000.0 mL |

The following examples are provided to illustrate the present invention and should not be construed as limiting the scope of the invention.

### EXAMPLE 1

### Production of Compounds I, II and III by fermentation

Fermentation conditions for the production of Compounds I, II, and III by the fungus Chaunopycnis pustulata. were as follows: vegetative mycelia of a culture of either of the above microorganisms were prepared by inoculating 54 mL of seed medium (Table 1) in a 250 mL unbaffled Erlenmeyer flask with frozen mycelia and conidia of MF5785 (ATCC74192) or MF6885 (ATCC PTA-4133). Seed flasks were incubated at 23° C. and 85% relative humidity on a rotary shaker with a 5-cm throw at 220 rpm in a room with constant fluorescent light, about 400 to 750 nm. Two-mL portions of the resulting 3-day culture were used to inoculate 50 mL portions of Liquid Production Medium (Table 2) in 250 mL unbaffled Erlenmeyer flasks; these cultures were incubated at 23° C., 220 rpm with 85% relative humidity in a room with constant fluorescent light for 14 days. The products appeared in the fermentation as early as 7 days with maximal accumulation observed at day 11. At harvest, the compounds were extracted as described in Example 2.

### EXAMPLE 2

Purification and Identification of Compounds I, II and III.

The instance compounds could be purified from crude extracts by a combination of chromatographic methods.

Fermentation broth prepared as described in Example 1, volume 2.6 liters, was exhaustively extracted with vigorous shaking with methyl ethyl ketone. After filtering and evaporating the extract under reduced pressure, the dried residue, amounting to 7.8 grams, was redissolved in 60 ml methylene chloride. A first fractionation was effected by column chromatography on 120 cc silica gel, eluting with 200 ml each methylene chloride and ethyl acetate. The latter contained the target compounds along with minor impurities and several other indole diterpenes, while many other impurities were removed by this step.

The ethyl acetate rich cut was evaporated down and partitioned between methanol and hexane to further eliminate impurities. A second step of purification was carried out by gel filtration on Sephadex LH-20 in methanol, affording the compounds at 0.8-1.0 column volumes of elution.

The resulting preparation was suitable for HPLC, which was carried out on a Zorbax RxC8 column (2.5×25 cm column, eluted at 8 ml/min with acetonitrile-water 50:50 (v/v) followed by a 100-minute gradient to 100% acetonitrile. Three fractions were collected:

Fraction A was further purified by HPLC on a 0.9×25 cm Phenomenex C8 column, eluting at 3 ml/min with a 100 minute gradient from 50% aqueous acetonitrile to 80% aqueous acetonitrile. This afforded 0.45 mg of pure Compound I after removing the solvent under reduced pressure and freeze-drying. Chromatographic characteristic: k′=13.8 on a 0.46×25 cm Zorbax RxC8 column maintained at 40° C. and eluted at 1 ml/min with a gradient of 30% to 100% acetonitrile in water over 30 minutes. Fraction B was similarly fractionated, yielding 0.6 mg Compound II.

Chromatographic characteristic: k′=14.3 on a 0.46×25 cm Zorbax RxC8 column maintained at 40° C. and eluted at 1 ml/min with a gradient of 30% to 100% acetonitrile in water over 30 minutes.

Fraction C was purified on the Phenomenex column as described above to afford 0.4 mg of Compound III. Chromatographic characteristic: Compound III: k′=15.3, measure taken on a 0.46×25 cm Zorbax RxC8 column maintained at 40° C. and eluted at 1 ml/min with a gradient of 30% to 100% acetonitrile in water over 30 minutes.

The purified compounds were identified by NMR and mass spectrometry.

Compound I: MW 617, C₃₇H₄₇NO₇; M+H obs. at 618.3403, calc. 618.3431. δ 1.17 (3H, s), 1.24 (3H, s), 1.31 (3H, s), 1.33 (3H, s), 1.34 (3H, s), 1.35 (3H, s), 1.36 (3H, s), 1.43 (3H, s), 1.57 (2H, m), 1.70 (1H, ddt), 1.87 (1H, dd), 1.98 (1H, ddt), 2.63 (1H, dd), 2.64 (1H, dd), 2.78 (1H, dd), 2.89 (1H, dd), 2.90 (1H, dd), 3.03 (1H, dd), 3.10 (1H, dd), 3.23 (1H, d), 3.31 (1H, dd), 3.91 (1H, d), 3.95 (1H, s), 4.11 (1H, d), 4.49 (1H, d), 5.41 (1H, dd), 6.91 (1H, d), 7.06 (1H, dd), 7.22 (1H, d), 7.77 (1H, br s).

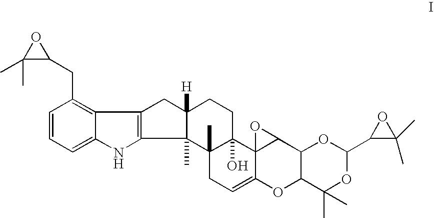

Compound II: MW 517, C₃₂H₃₉NO₅. M+H obs. at 518.2915; calc. 518.2906 δ 1.18 (3H, s), 1.23 (3H, s), 1.33 (3H, s), 1.42 (3H, s), 1.57 (2H, m), 1.69 (1H, br m), 1.72 (3H, s), 1.74 (3H, s), 1.89 (1H, dd), 1.99 (1H, m), 2.63 (1H, dd), 2.67 (1H, dd), 2.77 (1H, dd), 2.87 (1H, dd), 3.24 (1H, d), 3.88 (1H, d), 3.96 (1H, s), 4.10 (1H, d), 5.31 (1H, d of qt), 5.41 (1H, dd), 5.50 (1H, d), 7.10 (1H, m), 7.11 (1H, m), 7.35 (1H, dd), 7.44 (1H, dd) 7.76 (1H, br s)

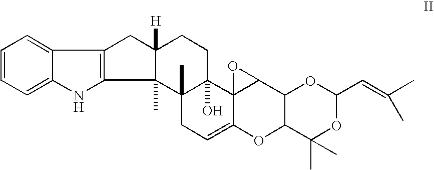

Compound III: MW 601, C₃₇H₄₇NO₆. M+H obs. at 602.3485, calc. 602.3482. δ 1.18 (3H, s), 1.24 (3H, s), 1.34 (3H, s), 1.35 (3H, s), 1.36 (3H, s), 1.43 (3H, s), 1.57 (2H, m), 1.68 (1H, br m), 1.77 (3H, s), 1.78 (3H, s), 1.87 (1H, dd), 1.98 (1H, ddt), 2.59 (1H, dd), 2.64 (1H, dd), 2.77 (1H, dd), 2.87 (1H, dd), 2.89 (1H, d), 3.24 (1H, d), 3.62 (1H, d), 3.89 (1H, d of qt), 3.91 (1H, d), 3.95 (1H, s), 4.11 (1H, d), 4.59 (1H, d), 5.40 (1H, br d), 5.41 (1H, dd), 6.88 (1H, d), 7.02 (1H, dd), 7.18 (1H, d), 7.69 (1H, br s).

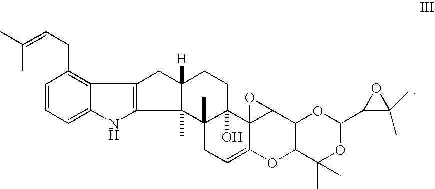

¹H NMR spectra were recorded at 500 MHz in CDCl₃ on a Unity 500 NMR spectrometer at 25° C. Chemical shifts are indicated in ppm relative to TMS at zero ppm using the solvent peak as internal standard. Only diagnostic peaks are noted. Abbreviations: s=singlet, d=doublet, q=quartet, br=broad, m=multiplet.

### EXAMPLE 3

Electrophysiological Effects on High-Conductance Calcium-Activated Potassium Channels

Patch clamp recordings of currents flowing through high-conductance calcium-activated potassium (maxi-K) channels were made from membrane patches excised from CHO cells constitutively expressing the α-subunit of the maxi-K channel and from HEK293 cells constitutively expressing both α- and β-subunits using conventional techniques (Hamill et al., 1981, Pflügers Archiv. 391, 85-100) at room temperature. Glass capillary tubing (Garner #7052) was pulled in two stages to yield micropipettes with tip diameters of approximately 1-2 microns. Pipettes were typically filled with solutions containing (mM): 150 KCl, 10 Hepes (4-(2-hydroxyethyl)-1-piperazine methanesulfonic acid), 1 MgCl₂, 0.01 CaCl₂, and adjusted to pH 7.20 with 3.7 mM KOH. After forming a high resistance (>109 ohms) seal between the plasma membrane and the pipette, the pipette was withdrawn from the cell, forming an excised inside-out membrane patch. The patch was excised into a bath solution containing (mM): 150 KCl, 10 Hepes, 5 EGTA (ethylene glycol bis(β-aminoethyl ether)-N,N,N′,N′-tetraacetic acid), sufficient Ca to yield a free Ca concentration of 1-5 μM, and the pH was adjusted to 7.2 with KOH. For example, 4.193 mM Ca was added to give a free concentration of 1 μM at 22° C. An EPC9 amplifier (HEKA Elektronic, Lambrect, Germany) was used to control the voltage and to measure the currents flowing across the membrane patch. The input to the headstage was connected to the pipette solution with a Ag/AgCl wire, and the amplifier ground was connected to the bath solution with a Ag/AgCl wire covered with a tube filled with agar dissolved in 0.2 M KCl. Maxi-K channels were identified by their large single channel conductance (˜250 pS) and sensitivity of channel open probability to membrane potential and intracellular calcium concentration.

Data acquisition was controlled by PULSE software (HEKA Elektronic) and stored on the hard drive of a MacIntosh computer (Apple Computers) for later analysis using PULSEFIT (HEKA Elektronic) and Igor (Wavemetrics, Oswego, OR) software.

Results:

The effects of the compounds of the present invention on maxi-K channels was examined in excised inside-out membrane patches. The membrane potential was held at −80 mV and brief voltage steps to positive membrane potentials (typically +50 mV) were applied once per 15 seconds to transiently open maxi-K channels. The fraction of channels blocked in each experiment was calculated from the reduction in peak current caused by application of 10 nM of specified compound to the internal side of the membrane patch. As a positive control in each experiment, maxi-K currents were eliminated at pulse potentials after the patch was transiently exposed to a low concentration of calcium (<10 nM) made by adding 1 mM EGTA to the standard bath solution with no added calcium.

| COMPOUND | FRACTION BLOCKED (10 nM) |
|---|---|
| Compound I | 0.9 |
| Compound II | 0.99-1 |
| Compound III | 0.99-1 |

After removal of compound from the bath, little or no recovery of peak current amplitude was observed in 20-40 minutes for all three compounds.

### EXAMPLE 4

The activity of the compounds can also be quantified by the following assay.

The identification of inhibitors of the Maxi-K channel is based on the ability of expressed Maxi-K channels to set cellular resting potential after transfection of both alpha and beta1 subunits of the channel in HEK-293 cells and after being incubated with potassium channel blockers that selectively eliminate the endogenous potassium conductances of HEK-293 cells. In the absence of maxi-K channel inhibitors, the transfected HEK-293 cells display a hyperpolarized membrane potential, negative inside, close to E_{K} (−80 mV) which is a consequence of the activity of the maxi-K channel. Blockade of the Maxi-K channel by incubation with maxi-K channel blockers will cause cell depolarization. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)).

Oxanol. is a lipophilic anion and distributes across the membrane according to membrane potential. Under normal conditions, when the inside of the cell is negative with respect to the outside, oxanol is accumulated at the outer leaflet of the membrane and excitation of coumarin will cause FRET to occur. Conditions that lead to membrane depolarization will cause the oxanol to redistribute to the inside of the cell, and, as a consequence, to a decrease in FRET. Thus, the ratio change (donor/acceptor) increases after membrane depolarization, which determines if a test compound actively blocks the maxi-K channel.

The HEK-293 cells were obtained from the American Type Culture Collection, 12301 Parklawn Drive, Rockville, Md., 20852 under accession number ATCC CRL-1573. Any restrictions relating to public access to the cell line shall be irrevocably removed upon patent issuance.

Transfection of the alpha and beta1 subunits of the maxi-K channel in HEK-293 cells was carried out as follows: HEK-293 cells were plated in 100 mm tissue culture treated dishes at a density of 3×10⁶ cells per dish, and a total of five dishes were prepared. Cells were grown in a medium consisting of Dulbecco's Modified Eagle Medium (DMEM) supplemented with 10% Fetal Bovine serum, 1X L-Glutamine, and 1×Penicillin/Streptomycin, at 37° C., 10% CO₂. For transfection with Maxi-K hα(pCIneo) and Maxi-K hβ1(pIRESpuro) DNAs, 150 μl FuGENE6™ was added dropwise into 10 ml of serum free/phenol-red free DMEM and allowed to incubate at room temperature for 5 minutes. Then, the FuGENE6™ solution was added dropwise to a DNA solution containing 25 μg of each plasmid DNA, and incubated at room temperature for 30 minutes. After the incubation period, 2 ml of the FuGENE6 ™/DNA solution was added dropwise to each plate of cells and the cells were allowed to grow two days under the same conditions as described above. At the end of the second day, cells were put under selection media which consisted of DMEM supplemented with both 600 μg/ml G418 and 0.75 μg/ml puromycin. Cells were grown until separate colonies were formed. Five colonies were collected and transferred to a 6 well tissue culture treated dish. A total of 75 colonies were collected. Cells were allowed to grow until a confluent monolayer was obtained. Cells were then tested for the presence of maxi-K channel alpha and beta1 subunits using an assay that monitors binding of ¹²⁵I-iberiotoxin-D19Y/Y36F to the channel. Cells expressing ¹²⁵I-iberiotoxin-D19Y/Y36F binding activity were then evaluated in a functional assay that monitors the capability of maxi-K channels to control the membrane potential of transfected HEK-293 cells using fluorescence resonance energy transfer (FRET) ABS technology with a VIPR instrument. The colony giving the largest signal to noise ratio was subjected to limiting dilution. For this, cells were resuspended at approximately 5 cells/ml, and 200 μl were plated in individual wells in a 96 well tissue culture treated plate, to add ca. one cell per well. A total of two 96 well plates were made. When a confluent monolayer was formed, the cells were transferred to 6 well tissue culture treated plates. A total of 62 wells were transferred. When a confluent monolayer was obtained, cells were tested using the FRET-functional assay. Transfected cells giving the best signal to noise ratio were identified and used in subsequent functional assays.

For Functional Assays:

The transfected cells (2E+06 Cells/mL) are then plated on 96-well poly-D-lysine plates at a density of about 100,000 cells/well and incubated for about 16 to about 24 hours. The medium is aspirated of the cells and the cells washed one time with 100 μl of Dulbecco's phosphate buffered saline (D-PBS). One hundred microliters of about 9 μM coumarin (CC₂DMPE)-0.02% pluronic-127 in D-PBS per well is added and the wells are incubated in the dark for about 30 minutes. The cells are washed two times with 100 μl of Dulbecco's phosphate-buffered saline and 100 μl of about 4.5 μM of oxanol (DiSBAC₂(3)) in (mM) 140 NaCl, 0.1 KCl, 2 CaCl₂, 1 MgCl₂, 20 Hepes-NaOH, pH 7.4, 10 glucose is added. Three micromolar of an inhibitor of endogenous potassium conductance of HEK-293 cells is added. A maxi-K channel blocker is added (about 0.01 micromolar to about 10 micromolar) and the cells are incubated at room temperature in the dark for about 30 minutes.

The plates are loaded into a voltage/ion probe reader (VIPR) instrument, and the fluorescence emission of both CC₂DMPE and DiSBAC₂(3) are recorded for 10 sec. At this point, 100 μl of high-potassium solution (mM): 140 KCl, 2 CaCl₂, 1 MgCl₂, 20 Hepes-KOH, pH 7.4, 10 glucose are added and the fluorescence emission of both dyes recorded for an additional 10 sec. The ratio CC₂DMPE/DiSBAC₂(3), before addition of high-potassium solution equals 1. In the absence of maxi-K channel inhibitor, the ratio after addition of high-potassium solution varies between 1.65-2.0. When the Maxi-K channel has been completely inhibited by either a known standard or test compound, this ratio remains at 1. It is possible, therefore, to titrate the activity of a Maxi-K channel inhibitor by monitoring the concentration-dependent change in the fluorescence ratio.

The IC50 activities of the maxi-K channel blockers in this assay ranged from about 0.5 nM to about 300 nM.

### EXAMPLE 5

Tremorgen Assay A mouse model was developed to evaluate the tremorgen potential of the instance compounds. Mice (CD-1) were injected intraperitoneally at 5 mg/Kg with each compound and observed by investigators for 1 hour for any signs of tremors as well as other behaviors not seen in control mice.

A known tremorgen, Aflatrem, consistently produces tremors in injected mice within 15 minutes at 1 and 5 mg/Kg. Test compounds injected at the same level, 5 mg/Kg, did not produce tremoring.

Table, Data for compounds tested for tremorgenic properties.

| Compound | Level Tested | Tremors |
|---|---|---|
| Compound I | 5 mg/Kg | None |
| Compound II | 5 mg/Kg | None |
| Compound III | 5 mg/Kg | None |
| Aflatrem | 1 and 5 mg/Kg | Tremors |
| Vehicle controls | | None |

1. A method for treating ocular hypertension or glaucoma which comprises administering to a patient in need of such treatment a therapeutically effective amount of a compound of Formula I, II or III: 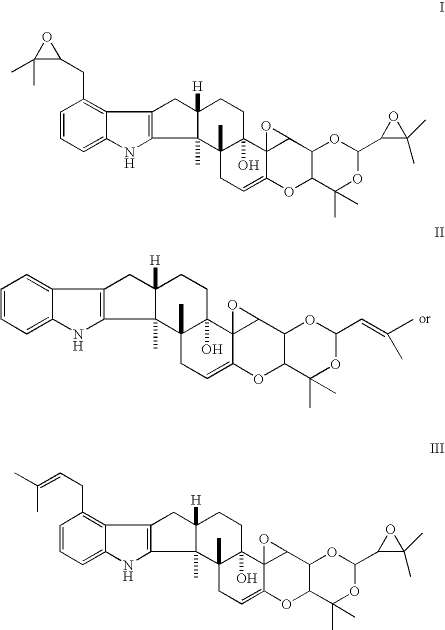
or a pharmaceutically acceptable salt, enantiomer, diastereomer or mixture thereof.

2. The method according to claim 1 wherein the compound of Formula I, II or III is administered as a topical formulation.

3. The method according to claim 2 wherein the topical formulation is a solution or suspension.

4. The method of claim 1, which comprises administering a second active ingredient, concurrently or consecutively, wherein the second active ingredient is selected from a β-adrenergic blocking agent, a parasympathomimetic agent, a carbonic anhydrase inhibitor, and a prostaglandin or a prostaglandin derivative.

5. The method according to claim 4 wherein the second active ingredient and the compound of formula I, II or III are administered as topical formulations.

6. The method according to claim 5 wherein the β-adrenergic blocking agent is timolol; the parasympathomimetic agent is pilocarpine; the carbonic anhydrase inhibitor is dorzolamide, acetazolamide, metazolamide or brinzolamide; the prostaglandin is latanoprost or rescula, and the prostaglandin derivative is a hypotensive lipid derived from PGF2α prostaglandins.

7. A method for treating macular edema or macular degeneration which comprises administering to a patient in need of such treatment a pharmaceutically effective amount of a compound of structural formula I, II or III: 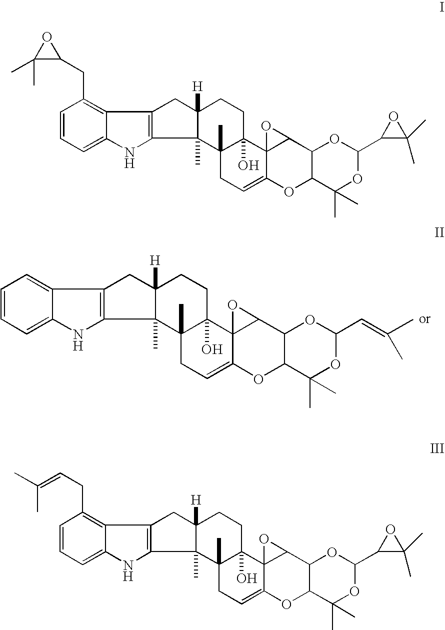
or a pharmaceutically acceptable salt, enantiomer, diastereomer or mixture thereof.

8. The method according to claim 7 wherein the compound of Formula I is applied as a topical formulation.

9. A method for increasing retinal and optic nerve head blood velocity or increasing retinal and optic nerve oxygen tension which comprises administering to a patient in need of such treatment a therapeutically effective amount of a compound of Formula I, II or III: 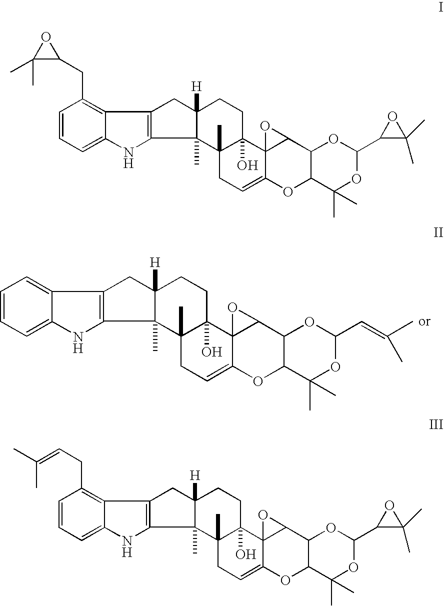
or a pharmaceutically acceptable salt, enantiomer, diastereomer or mixture thereof.

10. The method according to claim 9 wherein the compound of Formula I is applied as a topical formulation.

11. A method for providing a neuroprotective effect to a mammalian eye which comprises administering to a patient in need of such treatment a therapeutically effective amount of a compound of Formula I, II or III: 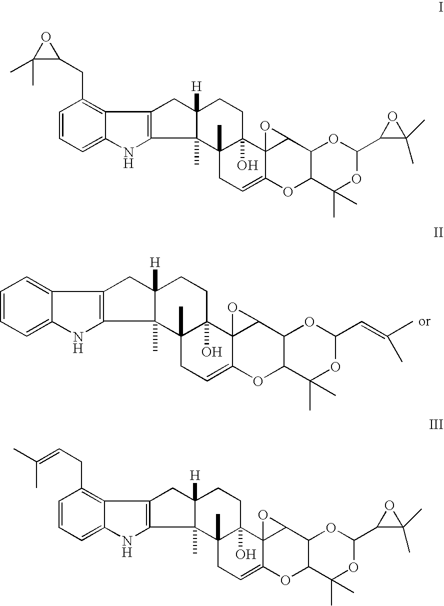
or a pharmaceutically acceptable salt, enantiomer, diastereomer or mixture thereof.

12. The method according to claim 11 wherein the compound of Formula I is applied as a topical formulation.

13. A method according to claim 2 in which the topical formulation contains xanthan gum or gellan gum.

14. A microbiological strain Chaunopycnis pustulata having the American Type Culture Collection number PTA-4133, MF6885.

15. A method of making the compounds of claim 1 using microbiological strain Chaunopycnis pustulata, having the American Type Culture Collection number PTA-4133, MF6885.
